(19) United States
(12) Patent Application Publication
(10) Pub. No.: US 2026/0273043 A1
(43) Pub. Date: Sep. 17, 2026

(54) CHIMERIC HUMAN RECEPTOR FOR PATHOGENIC VIRUSES

(71) Applicant: MARY HITCHCOCK MEMORIAL HOSPITAL, FOR ITSELF AND ON BEHALF OF DARTMOUTH-HITCHCOCK CLINIC, Lebanon, NH (US)

(72) Inventor: Richard I. ENELOW, Lebanon, NH (US)

(21) Appl. No.: 19/124,532

(22) PCT Filed: Oct. 30, 2023
(86) PCT No.: PCT/US2023/036254

§ 371 Date: Apr. 25, 2025

Related U.S. Application Data
(60) Provisional application No. 63/420,461, filed on Oct. 28, 2022.

(51) Int. Cl. A61K 39/215; A61K 39/00; A61K 48/00; A61P 37/04; C07K 14/705

(52) U.S. Cl. CPC A61K 39/215; A61K 48/0058; A61P 37/04; C07K 14/70596; A61K 2039/57

(57) ABSTRACT

A chimeric protein encoded by an expression plasmid is provided. The chimeric protein comprises an extracellular of a pathogen-binding receptor and an intracellular domain of an innate immune signaling protein, such as the TLR family of receptors.

### CROSS-REFERENCE TO RELATED APPLICATION

This application is a United States National Stage Entry of International Patent Application No. PCT/US2023/036254, filed on Oct. 30, 2023; which claims benefit of priority to U.S. Provisional Patent Application No. 63/420,461 filed on Oct. 28, 2022, the entire content of the aforementioned applications is incorporated herein by reference in its entirety.

### SEQUENCE LISTING

The instant application contains a Sequence Listing which has been submitted herewith and is hereby incorporated by reference in its entirety. Said .xml copy, created on May 13, 2026 is named DRTHP2003US00638571, and is 8,607 bytes in size.

### BACKGROUND

Pathogenic viruses enter human cells by binding to a specific receptor, triggering various biologic activities leading to internalization, uncoating of the virus capsid, and replication of the virus by transcription of viral nucleic acids, and translation of viral proteins. Animal cells have evolved a multitude of innate defense mechanisms over millennia to counter the intrusion of viral genomes and the hijacking of cellular protein synthetic machinery. In parallel fashion, essentially every virus not lost to natural selection has evolved numerous biologic countermeasures which rapidly suppress/evade these host antiviral tactics, via all manner of distinct mechanisms (“convergent evolution”), with varying degrees of efficiency.

The most highly pathogenic viruses are usually the most efficient at such countermeasures, notably Ebola, avian influenza, and many of the coronaviruses (particularly SARS-COV, MERS, and SARS-COV-2). SARS-COV-2, for example, has such a “refined and thorough” panoply of countermeasures that in the early stages of infection (often as long as 4-5 days), the virus is capable of replicating with such stealth that essentially no symptoms arise (which are caused by the so-called “innate immune responses”) in the infected individual, while transmission to others during this period may be rampant.

The innate cellular antiviral immune responses are responsible for (A) the inflammation, swelling, and mucous production which sets up an “advantageous battleground” for definitive host immune responses (and which result in the typical symptoms of an upper respiratory infection); (B) the critical activation of “adaptive immune responses”, namely T cell and B cell responses, which can take 4-5 days (or longer) to develop and which are required to actually clear the pathogen; and C) putting “brakes” on viral replication, thereby slowing viral growth and the spread to neighboring cells, buying time for the adaptive responses activate, develop, and deploy. The faster the adaptive responses can robustly activate and deploy, the more limited the number of host cells will harbor virus, and the more limited the tissue damage which will accompany immune-mediated virus clearance.

A rapid and robust adaptive immune activation also results in robust immune memory, and protection from re-infection. In stark contrast the near-complete suppression of early innate responses by highly efficient viral countermeasures results in (A) unimpeded, asymptomatic viral replication, allowing spread throughout the respiratory tract (including lower gas exchange units), as well as spread to other individuals; (B) delayed activation of adaptive immune response such that the distal airspaces (alveoli) become the site of the battle between host and the widely-distributed infection; (C) massive immune-mediated damage to the gas-exchange units of the distal respiratory tract; and (D) relatively weak memory/recall responses, permitting frequent reinfection (i.e., weak secondary immunity).

Not only are primary adaptive immune responses slow to mobilize without innate immune activation, “secondary” adaptive (vaccine/memory) responses are proportionately slower. Thus, the enhanced kinetics and magnitude of a strong secondary response is usually adequate to eliminate viral spread throughout the airways prior to encroachment into the gas exchange units of the distal lung (thus preventing serious illness), but often still sluggish enough to permit initiation of infection. The impressive benefits conferred by the remarkable vaccines to SARS-COV-2 still require some degree of innate immune activation to be awakened into action, and thus transmission may still occur. Respiratory viral replication, even in the absence of serious illness, may allow transmission and thus poses the threat of new variant development, which may ultimately evade our vaccine responses.

### SUMMARY

Disclosed are chimeric receptors designed to circumvent viral evasion strategies. In one embodiment, these chimeric receptors contain the extracellular and transmembrane domains of receptors to which pathogens bind, and the extracellular and transmembrane domains are linked to the intracellular domain of an innate immune signaling receptor, such as toll-like receptor 3 (TLR3). The chimeric receptor of the present disclosure selectively triggers the innate immune gene expression program immediately and exclusively upon binding by a pathogen at the cell surface.

In an aspect, the present disclosure comprises a chimeric protein comprising: a) an extracellular (EC) domain and transmembrane domain; b) an intracellular (IC) domain of an innate immune signaling protein or an intracellular (IC) domain of a protein capable of signaling the recognition of a pathogen-associated molecular domain (PAMP) and activating innate immune gene expression; and c) a linker positioned between (a) and (b), the linker permitting signal transduction from the EC domain to the IC domain, hereby activating innate immune gene expression. In one aspect, the EC domain is capable of binding to a pathogen. In another aspect, the EC domain and the IC domain are not from the same naturally existing protein, or in other words, these domains are either synthetic (i.e., man-made) or they are from different proteins that exist in nature. In another aspect, the transmembrane domain and the EC domain of the chimeric protein are from same protein. In another aspect, the transmembrane domain and the EC domain of the chimeric protein are not from same protein.

In an embodiment, the innate immune signaling protein is a member of TLR family of receptors. In one aspect, the TLR family of receptors are typically directly activated by PAMPS indicating the presence of an RNA virus. In another embodiment, the innate immune signaling protein is a toll-like receptor selected from TLR3 and TLR7, or other family member of toll-like receptors, including but not limited to TLR2, TLR4, TLR5, TLR8. In embodiment, the pathogen-binding receptor binds a respiratory pathogen, or a pathogen that is transmitted via the respiratory tract. In another aspect, the pathogen is a pathogen belonging to a family selected from the group consisting of hantaviridae, paramyxoviridae, picornoviridae. In another aspect, the pathogen is a coronavirus selected from the group consisting of SARS-COV-2 and MERS.

In an embodiment, a polynucleotide encodes the chimeric protein of the present disclosure. In another embodiment, the EC domain of the chimeric protein is at least 90%, or 95%, or 99% identical in amino acid sequence to the EC domain of human ACE2 protein, and the EC domain binds to spike protein of SARS-COV-2. In another embodiment, the IC domain is at least 90%, or 95%, or 99% identical in amino acid sequence to the IC domain of human TLR3 protein and the IC domain activates innate immune gene expression. In another embodiment, the linker domain is at least 90%, or 95%, or 99%, or is 100% identical in amino acid sequence to a sequence selected from the group consisting of SEQ ID NO: 3 and SEQ ID NO: 4. In another embodiment, the chimeric protein is at least 90%, or 95%, or 99%, or is 100% identical in amino acid sequence to SEQ ID NO: 1.

In an embodiment, an expression plasmid contains one or more polynucleotide molecules of the present disclosure, and these polynucleotides may be DNA or mRNA molecules. In another embodiment, the polynucleotide has a nucleotide sequence that is 90%, or 95%, or 99%, or is 100% identical to nucleotide sequence of SEQ ID NO: 2. In an embodiment, the expression plasmid further comprises a promoter, said promoter regulating expression of the chimeric protein. By way of example, the promoter may regulate specific expression of the chimeric protein when the expression vectors enter cells of the respiratory system, or more specifically, cells of the respiratory tract. In an embodiment, the expression plasmid further comprises features which may enhance eukaryotic expression, including, but not limited to, a suitable promoter (such as CMV), a poly A tail, a Kozak sequence, a DYK tag, and other components.

In another aspect, the present disclosure provides a method for selectively triggering innate immune gene expression. In an embodiment, the method comprises administering to a subject a composition containing an expression plasmid (or expression vector) or mRNA encoding the chimeric protein, wherein after administration, the chimeric protein is expressed as a transmembrane protein on the cell surface, wherein, upon exposure to a pathogen, the pathogen binds to the extracellular domain of the chimeric protein and triggers toll-like receptor (e.g., TLR3) signaling. In one aspect, the chimeric protein is expressed as a transmembrane protein on the cell surface of the respiratory tract. In an embodiment, the expression vector contains a polynucleotide that has a nucleotide sequence that is 90%, or 95%, or 99%, or is 100% identical to nucleotide sequence of SEQ ID NO: 2. In another embodiment, the expression vector contains a DNA or an mRNA encoding a chimeric protein having at least 90%, or 95%, or 99%, or is 100% sequence identity in amino acid sequence to SEQ ID NO: 1.

In an embodiment, the expression plasmid or mRNA is administered intra-nasally or through a part of the respiratory tract in a form of nebulization. In an embodiment, the expression plasmid or mRNA is encapsulated in a lipid particle (LNP) or a liposome for mucosal delivery. In one aspect, the composition further comprises an ingredient that stabilizes the DNA or mRNA. In another aspect, the composition further comprises an ingredient that helps the DNA or mRNA enter into cells of the subject. In another aspect, the ingredient specifically helps the DNA or mRNA enter into specific cell types of the subject, for example, cells of the respiratory tract.

### BRIEF DESCRIPTION OF THE DRAWINGS

FIG. 1 shows an exemplary embodiment of the chimeric virus receptor of the present disclosure. ACE2 extracellular and transmembrane domains are linked to the intracellular domain of the innate immune signaling receptor TLR3 and expressed as homodimers on the cell surface.

FIG. 2 demonstrates that the chimeric molecules of the present invention may be expressed on the cell surface and can transduce a signal upon ligand binding.

FIG. 3 shows the kinetics of IFNβ production by VSV-spike pseudovirus expressing the SARS-COV-2 spike protein) binding to fusion receptor construct in HEK293 cells.

FIG. 4 shows protein sequence of ACE2.linker2. TLR3 (SEQ ID NO:1) and polynucleotide sequence (SEQ ID NO:2) encoding this protein, as well as examples of two linker sequences: Linker 1 (SEQ ID NO:3) and Linker 2 (SEQ ID NO:4).

FIG. 5 shows fluorescence imaging of Calu-3 cells, which support infection with a wild-type replication-competent (rc) VSV-spike expressing GFP (FIG. 5B, compared with uninfected cells in FIG. 5A), due to native ACE2 receptor expression, though the amount of green fluorescence (rcVSV particles) is higher in cells transfected with a control ACE2 vector (FIG. 5C). The green fluorescence (viral load) in cells transfected with the chimeric receptor (FIG. 5D) is significantly lower than the fluorescence observed in the untransfected cells subjected to the same rcVSV-spike-GFP virus infection (FIG. 5B). Images were acquired at 48 hours post-infection.

### DETAILED DESCRIPTION

The present disclosure relates to a chimeric receptor encoded by an expression plasmid by joining the cDNA sequence for extracellular and transmembrane domains of pathogen-binding receptors with the intracellular domain of an innate immune signaling receptor, such as TLR3.

Several attempts have been made to use locally delivered innate immune stimulants, including intranasal interferon, artificial double-stranded RNA, or other mimics of pathogen-associated molecular patterns (PAMPS). The novelty of this invention is the ability to enable selective activation of innate immune signaling exclusively in those cells subjected to actual infection, and thus avoiding non-selective widespread activation throughout the upper respiratory tract and the associated toxicity. Thus, the utility of the present disclosure extends to immuno-prophylaxis, to early intervention, and to novel vaccination strategies with inactivated (harmless) pathogens.

With or without widespread dissemination of effective vaccines, and frequent booster administration, the ideal of inducing innate responses in initial stages of infection, whether to SARS-COV-2 (or to any novel variant), to the next coronavirus that will undoubtedly emerge (such as MERS, or any of a multitude of novel strains currently residing in the bat population), or to any emerging member of the hantaviridae, paramyxoviridae, picornoviridae family, or a highly pathogenic influenza, such as avian H5N1, is undeniable yet difficult to achieve. One strategy might be found in the administration of type I interferon (IFN-alpha,-beta,-lambda) each of which is already used in very different, limited clinical circumstances, and represent the pharmacologic delivery of a potent “innate immune response”. Several trials of administration in Covid-19 patients demonstrate the profound limitations of this strategy. First, delivery to patients after development of illness results in overactivation of destructive immune activity (poor timing; poor location), as well as the (not unexpected) toxicity of pharmacologic level of administration, in a widespread fashion. There is the theoretic advantage imaginable in administration of a small dose, for example, exclusively to the upper respiratory tract, such as the nasal passages (immediately upon a known exposure), though there is still considerable local toxicity, as well the logistical challenges of such a strategy. While not as widespread, this type of local administration would still be untargeted.

This disclosure describes a potentially more refined approach to circumventing viral evasion strategies, using the timely example of SARS-COV-2 infection, the receptor for which is cell surface ACE2 (angiotensin converting enzyme 2). The ACE2 molecule has a large extracellular domain (to which the SARS-COV-2 spike protein binds), a transmembrane domain, and a short intracellular domain (the function of which is unknown). In one embodiment of this invention, a chimeric receptor is encoded by an expression plasmid by joining the cDNA sequence for the ACE2 extracellular (EC) and transmembrane (TM) domains with the intracellular (IC) domain of the innate immune signaling receptor TLR3 (toll-like receptor 3) in substitution for the IC domain of ACE2 (with a short linker sequence to separate the domains).

In one aspect, the invention is designed to selectively trigger the innate immune antiviral host gene expression program, naturally induced by TLR3 receptor signaling immediately and exclusively upon binding of the heterologous ACE-2 EC domain by SARS-COV-2 at the cell surface. Thus, no such signals will be transduced in any cells by this chimeric receptor in the absence of binding/engagement of the SARS-COV-2 virion (i.e., upon cellular infection). It should be noted that the “physiologic” ligand for induction of TLR3 signaling is double-stranded RNA (dsRNA), which is never found in an animal (eukaryotic) cell except in one that is infected with a dsRNA virus or ssRNA virus (ssRNA is transcribed into dsRNA to provide a reverse template for RNA polymerase-mediated RNA replication). TLR3 is one of a family of sensors of “foreign patterns” (so-called pathogen-associated molecular patterns, or PAMPS), sensing of which triggers expression of multiple cellular gene products which disrupt and interfere with viral machinery. Hence the translation of the viral proteins which efficiently impede host antiviral mechanisms is delayed (relative to the initial entry of virus), and requires transcription and translation of viral proteins, such that the rate of induction of host innate antiviral responses is a critical determinant of how much and how long the virus may replicate in “immunologic silence”, and thus determines the probability of the virus escaping cellular inhibition, massively replicating, and accumulating a high viral load in the upper airway leading readily to transmission and spread (as well as potentially making its way to the distal lung with a large viral load).

During the various waves of the Covid-19 pandemic, even prior to vaccine availability, the probability of any infected individual succumbing to severe Covid-19 has been generally low (and younger members of the population appear to have an advantage in rate of innate antiviral activation, perhaps due to the increased frequency of recent experience with prior respiratory virus infection). It is also well known that older individuals tend to mount relatively weak innate responses and make weaker vaccine responses (such as observed after influenza vaccination in older populations).

There are a number of potential applications of the chimeric platform, whether utilizing the extracellular domain of the ACE2 receptor, or the DPP4 receptor (for MERS), the ephrin-B2 (for Nipah virus, currently spreading in humans in south Asia), or receptors for other pathogen families which may threaten the human species. For example, prophylactic intranasal administration to an entire student body prior to arrival on a campus, to health care workers entering a zone of high transmission, or to military personnel entering a theater where biologic weapons may be encountered, and other such scenarios are easily imagined. In addition, widespread screening and early detection of asymptomatic cases, or contacts thereof, presents a unique opportunity to both protect against severe infection, as well as provide effectively “emergent vaccination” to such individuals as they are detected.

The duration of immunoprophylactic protection after administration is likely to be on the order of 1-3 weeks. The potential use in prophylaxis against infection might pertain, for example, to administration to students immediately prior to arrival on a campus, to emergency medical workers responding to an outbreak of a novel pathogen (e.g. Nipah, Hanta, or a new coronavirus), or in conjunction with routine screening of asymptomatic individuals in the setting of a pandemic (with immediate administration to those who test positive). Each of these examples illustrates potential utility without re-administration (though this is an additional option to be explored, depending upon the anticipated application). This presents the opportunity for rapid deployment after detection of a novel pathogen, prior to vaccine development and distribution to military personnel entering a theater in which biologic weapons may be encountered, or other urgencies in which chimeric constructs (using various well-known pathogen receptors “mounted” on the platform) might be stockpiled in quantities that do not require unusually stringent (e.g., −80° C.) storage and transport conditions.

Finally, the opportunity for a non-emergent and extremely safe vaccine strategy for novel pathogens is envisioned by the administration of the chimeric platform, followed immediately by an inactivated/killed pathogen of any variety (rapid generation of which can be accomplished with little more than a sequence). By virtue of the innate activation provided by the invention, robust antibody (B cell) and T cell responses (CD4+ and CD8+) will be generated, via mechanisms already well-understood. Importantly the immunization will be generated at the mucosal surface, which is the natural entrance point for most of these pathogens. Such an immunization strategy may also be a useful method for immunizing against pathogens already in circulation, but for which vaccine efforts have been challenging.

The term “innate immune signaling molecule” refers to signaling molecules involved in detecting pathogen (PAMPS) and activating the body's innate immune response within hours or a few days of pathogen invasion, which is much faster than the body's other immune response (called adaptive immune response) involving T and B cells and typically takes one week or longer to become activated. Detailed description of examples of innate immune signaling molecules and activation of innate immune gene expression can be found in Cui et al., “Mechanisms and pathways of innate immune activation and regulation in health and cancer” Hum Vaccin Immunother. 2014 November; 10 (11): 3270-3285. See also, Daimond, et al., Nature Immunology, 23:165-76 (2022); and Cassetti, et al., J of Infectious Disease, 2023:227, 1433-41 (2023).

The present disclosure is further illustrated but not limited by the following items:
Item 1: A polynucleotide encoding a chimeric protein, the chimeric protein comprising:
   a) an extracellular (EC) domain and transmembrane domain, wherein said EC domain is capable of binding to a pathogen;
   b) an intracellular (IC) domain of an innate immune signaling protein or an intracellular (IC) domain of a protein capable of signaling the recognition of a pathogen-associated molecular domain (PAMP) and activating innate immune gene expression; and
   c) a linker positioned between (a) and (b), said linker permitting signal transduction from the EC domain to the IC domain, hereby activating innate immune gene expression.
Item 2: The polynucleotide of Item 1, wherein the innate immune signaling protein is a member of TLR family of receptors.
Item 3: The polynucleotide of any preceding items, wherein the innate immune signaling protein is a member selected from the group consisting of TLR3 and TLR7.
Item 4: The polynucleotide of any preceding items, wherein the pathogen is a pathogen transmitted via the respiratory tract.
Item 5: The polynucleotide of any preceding items, wherein the pathogen is a pathogen belonging to a family selected from the group consisting of hantaviridae, paramyxoviridae, picornaviridae,

Item 6: The polynucleotide of any preceding items, wherein the pathogen is a coronavirus selected from the group consisting of SARS-COV-2 and MERS (or any of a multitude of novel strains currently residing in the bat population).
Item 7: The polynucleotide of any preceding items, wherein the EC domain is at least 95% identical in amino acid sequence to the EC domain of human ACE2 protein, and the EC domain binds to spike protein of SARS-COV-2.
Item 8: The polynucleotide of any preceding items, wherein the IC domain is at least 95% identical in amino acid sequence to the IC domain of human TLR3 protein and the IC domain activates innate immune gene expression.
Item 9: The polynucleotide of any preceding items, wherein the linker domain is a sequence selected from the group consisting of SEQ ID NO: 3 and SEQ ID NO: 4.
Item 10: The polynucleotide of any preceding items, wherein the EC domain and the IC domain are not from the same naturally existing protein.
Item 11: The polynucleotide of any preceding items, wherein the chimeric protein is at least 95% identical in amino acid sequence to SEQ ID NO: 1.
Item 12: The polynucleotide of any preceding items, wherein the chimeric protein has an amino acid sequence to SEQ ID NO: 1.
Item 13: The polynucleotide of any preceding items, wherein the polynucleotide is a DNA or an mRNA.
Item 14: The polynucleotide of any preceding items having a nucleotide sequence that is at least 95% identical to nucleotide sequence of SEQ ID NO: 2.
Item 15: The polynucleotide of any preceding items having a nucleotide sequence that is identical to nucleotide sequence of SEQ ID NO: 2.
Item 16: An expression vector comprising the polynucleotide of any preceding items.
Item 17: The expression vector of Item 16, further comprising a promoter, wherein the promoter regulates expression of the chimeric protein.
Item 18: A chimeric protein comprising
   a) an extracellular (EC) domain and transmembrane domain, wherein said EC domain is capable of binding to a pathogen;
   b) an intracellular (IC) domain of an innate immune signaling protein or an intracellular (IC) domain of a protein capable of signaling the recognition of a pathogen-associated molecular domain (PAMP) and activating innate immune gene expression; and
   c) a linker positioned between (a) and (b), said linker permitting signal transduction from the EC domain to the IC domain, hereby activating innate immune gene expression,
   wherein the EC domain and the IC domain are not from the same naturally existing protein.
Item 19: A method for triggering innate immune response, comprising
   a) administering to a subject a composition comprising an expression vector comprising the polynucleotide of any of Items 1-15, wherein after administration, the chimeric protein is expressed as a transmembrane protein on cell surface of certain cells of the subject, and
   b) allowing the extracellular domain of the chimeric protein to bind to a pathogen to which the subject is exposed, thereby triggering innate immune response.
Item 20: The method of Item 19, wherein the composition is administered intra-nasally or through a part of the respiratory tract in a form of nebulization

Item 21: The method of any of Items 19-20, wherein the composition is administered to the subject in a lipid particle (LNP).
Item 22: The method of any of Items 19-21, wherein the polynucleotide has a nucleotide sequence of SEQ ID NO: 2.
Item 23: The method of any of Items 19-22, wherein the polynucleotide encodes a chimeric protein having an amino acid sequence of SEQ ID NO: 1.
Item 24: The method of any of Items 19-23, wherein the polynucleotide is a DNA or an mRNA and the composition further comprises an ingredient that stabilizes the DNA or mRNA.
Item 25: The method of any of Items 19-24, wherein the composition further comprises an ingredient that helps the DNA or mRNA enter into cells of the subject.

It will be readily apparent to those skilled in the art that the methods described herein may be modified and substitutions may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

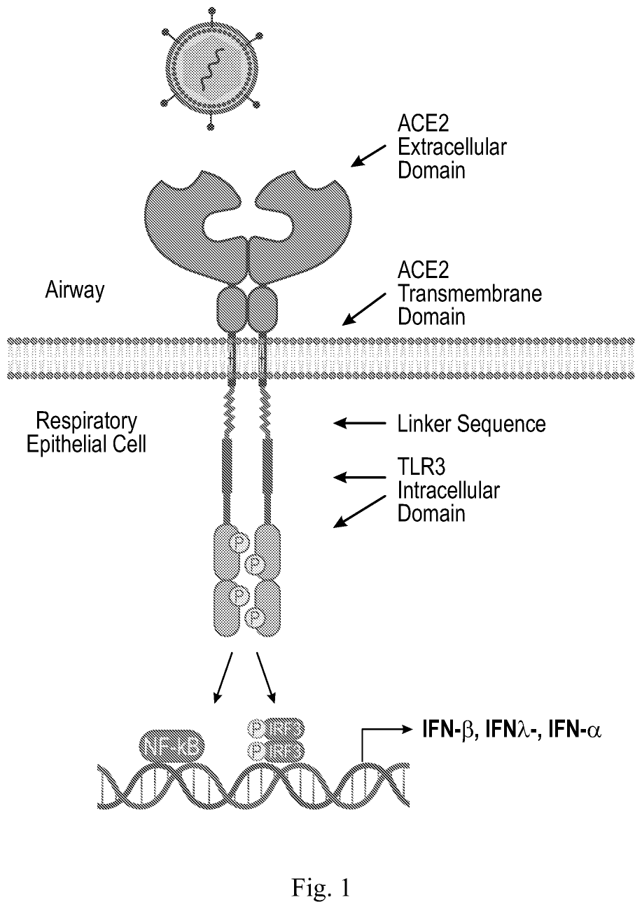

The timely example of the SARS-COV-2 receptor, (ACE2; angiotensin converting enzyme 2) is shown herein as proof of principle. The ACE2 molecule has a large extracellular domain (to which the SARS-COV-2 spike protein binds), a transmembrane domain, and a short intracellular domain (the function of which is unknown). An embodiment of present disclosure includes, but is not limited to, a chimeric receptor encoded by an expression plasmid joining the cDNA sequence for the ACE2 extracellular and transmembrane domains with the intracellular domain of the innate immune signaling receptor TLR3, with a short linker sequence (FIG. 1).

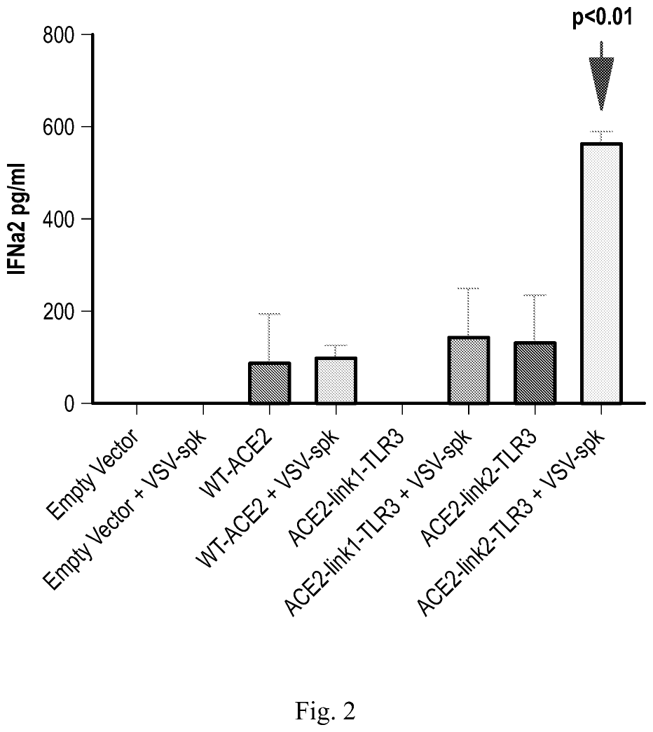

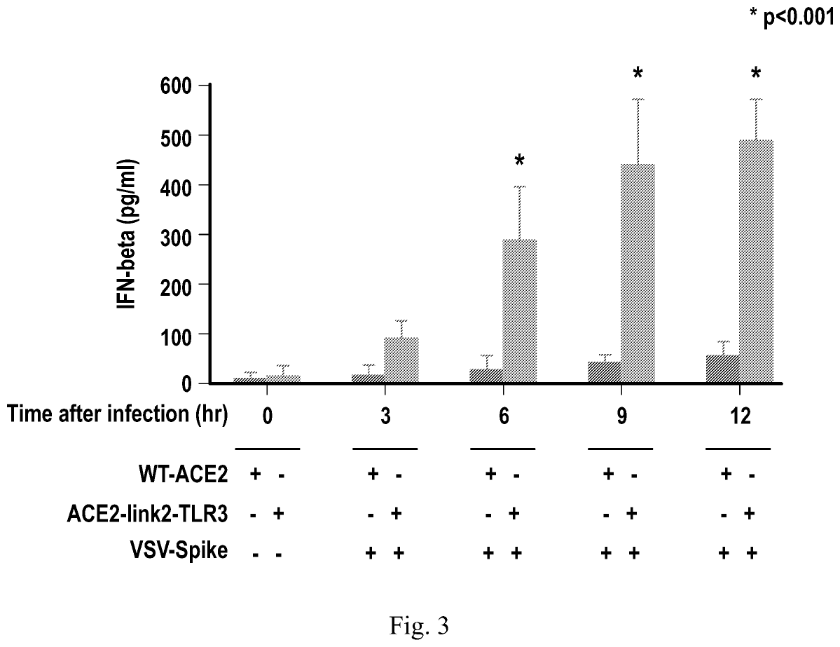

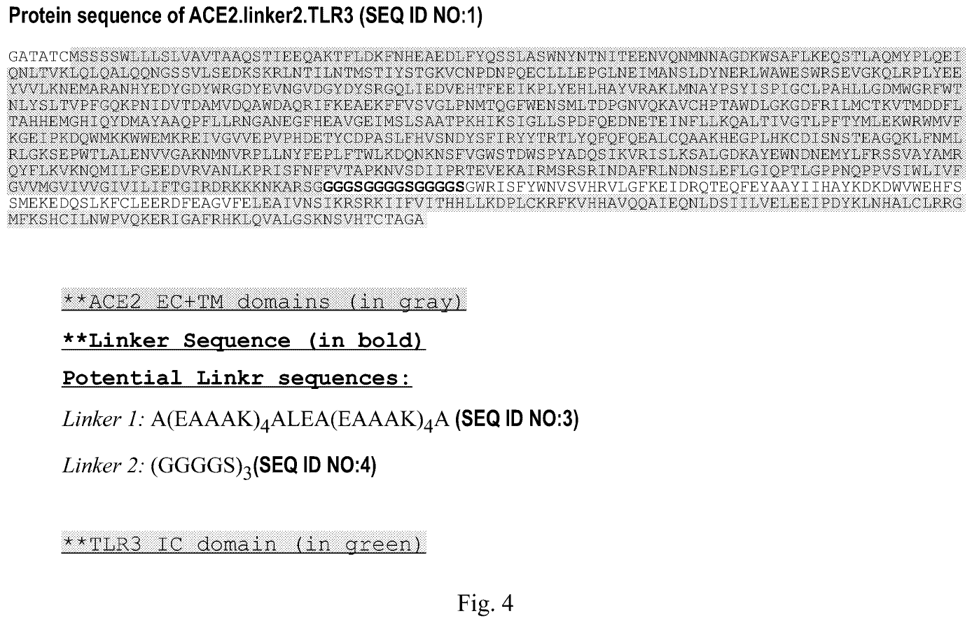

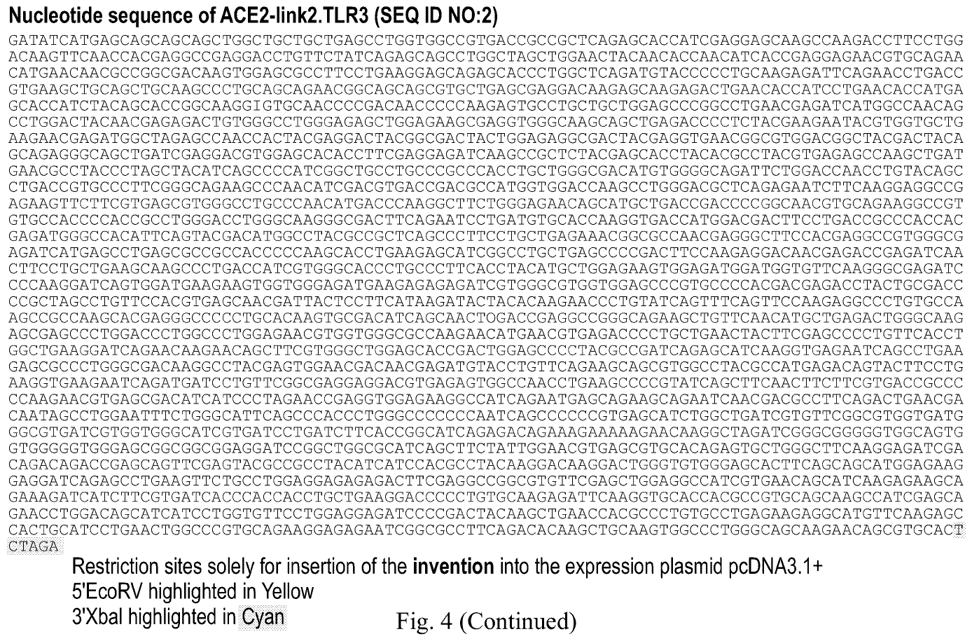

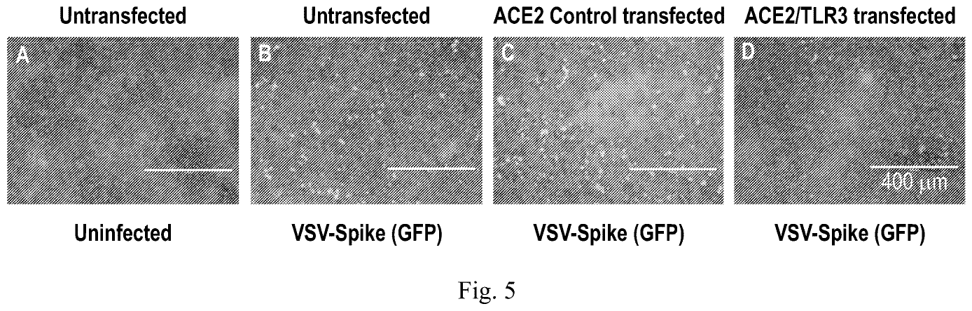

To determine if the chimeric molecule can be expressed on the cell surface and can transduce a signal upon ligand binding, several variations of the invention were designed using different linker sequences. The ligand used to test the signaling capacity is a (replication-incompetent; pseudotyped) vesicular stomatitis virus (VSV) engineered to express the SARS-COV-2 spike protein (so called VSV-spike pseudotyped virus, which can be used without the biocontainment requirements for working with SARS-COV-2). As shown in FIG. 2, HEK293 cells (a commonly used human epithelial cell line) were transiently transfected with one of 4 expression vectors (cDNA): (A) an empty vector control (without a cDNA insert); (B) a vector containing a cDNA for wildtype ACE2; (C) a vector containing a version of the chimeric receptor with one form of the linker sequence; and (D) a vector containing a different version of the chimeric receptor with a different form of linker sequence. Shown are levels of type I IFN production into the media (by ELISA) 12 hours after addition of the VSV-spike pseudotyped virus (versus no virus added, as shown). The significant production of IFNα2 at 12 hours is indicative of robust signaling by the chimeric receptor (with linker sequence 2), and the magnitude of innate IFN production indicates the feedforward effect of early IFNβ induction of IFNα. This is evident (as shown in FIG. 3) by the kinetics of IFNβ production, considerably more rapid after ligation of the chimeric receptor, indicating that the signal is transduced immediately upon virus binding. The demonstration of direct activation of innate immune gene expression triggered by the initial ligation of cell surface receptor by a virus particle indicates that this mechanism will supercede the suppressive mechanisms of any virus whose evasion strategies require internalization, transcription, and translation of viral proteins which function in host cell immune suppression. This is phenotypically exemplified by an experiment using an airway epithelial cell line (Calu-3, which expresses native ACE2 receptor), transfected with the chimeric receptor (or with a wild-type control ACE2 expression vector, otherwise identical to the vector expressing the chimeric receptor). As shown in FIG. 5, untransfected Calu-3 cells support infection with a wild-type replication-competent (rc) VSV-spike expressing GFP (FIG. 5B, compared with uninfected cells in FIG. 5A) due to native ACE2 receptor expression, though the amount of green fluorescence (rcVSV particles) is significantly higher in cells transfected with a control ACE2 vector (FIG. 5C). However, the green fluorescence (viral load) in cells transfected with the chimeric receptor (FIG. 5D) is significantly lower than the fluorescence in the untransfected cells subjected to the same rcVSV-spike-GFP virus infection (FIG. 5B). Images were acquired at 48 hours post-infection.

The contents of all cited references (including literature references, patents, patent applications, and websites) that are cited throughout this application are hereby expressly incorporated by reference in their entirety for any purpose into the present disclosure. The disclosure may employ, unless otherwise indicated, conventional techniques of microbiology, molecular biology and cell biology, which are well known in the art.

The disclosed methods may be modified without departing from the scope hereof. It should be noted that the matter contained in the above description or shown in the accompanying drawings should be interpreted as illustrative and not in a limiting sense.

What is claimed is:

1. A polynucleotide encoding a chimeric protein, the chimeric protein comprising:
a) an extracellular (EC) domain and transmembrane domain, wherein said EC domain is capable of binding to a pathogen;
b) an intracellular (IC) domain of an innate immune signaling protein or an intracellular (IC) domain of a protein capable of signaling the recognition of a pathogen-associated molecular domain (PAMP) and activating innate immune gene expression; and
c) a linker positioned between (a) and (b), said linker permitting signal transduction from the EC domain to the IC domain, hereby activating innate immune gene expression.

2. The polynucleotide of claim 1, wherein the innate immune signaling protein is a member of TLR family of receptors.

3. The polynucleotide of claim 2, wherein the innate immune signaling protein is a member selected from the group consisting of TLR3 and TLR7.

4. The polynucleotide of claim 1, wherein the pathogen is a pathogen transmitted via the respiratory tract.

5. The polynucleotide of claim 1, wherein the pathogen is a pathogen belonging to a family selected from the group consisting of hantaviridae, paramyxoviridae, picornoviridae.

6. The polynucleotide of claim 1, wherein the pathogen is a coronavirus selected from the group consisting of SARS-COV-2 and MERS.

7. The polynucleotide of claim 1, wherein the EC domain is at least 95% identical in amino acid sequence to the EC domain of human ACE2 protein, and the EC domain binds to spike protein of SARS-COV-2.

8. The polynucleotide of claim 1, wherein the IC domain is at least 95% identical in amino acid sequence to the IC domain of human TLR3 protein and the IC domain activates innate immune gene expression.

9. The polynucleotide of claim 1, wherein the linker domain is a sequence selected from the group consisting of SEQ ID NO: 3 and SEQ ID NO: 4.

10. The polynucleotide of claim 1, wherein the EC domain and the IC domain are not from the same naturally existing protein.

11. The polynucleotide of claim 1, wherein the chimeric protein is at least 95% identical in amino acid sequence to SEQ ID NO: 1.

12. The polynucleotide of claim 1, wherein the chimeric protein has an amino acid sequence to SEQ ID NO: 1.

13. The polynucleotide of claim 1, wherein the polynucleotide is a DNA or an mRNA.

14. The polynucleotide of claim 1 having a nucleotide sequence that is at least 95% identical to nucleotide sequence of SEQ ID NO: 2.

15. The polynucleotide of claim 1 having a nucleotide sequence that is identical to nucleotide sequence of SEQ ID NO: 2.

16. An expression vector comprising the polynucleotide of claim 1.

17. The expression vector of claim 16, further comprising a promoter, wherein the promoter regulates expression of the chimeric protein.

18. A chimeric protein comprising:
a) an extracellular (EC) domain and transmembrane domain, wherein said EC domain is capable of binding to a pathogen;
b) an intracellular (IC) domain of an innate immune signaling protein or an intracellular (IC) domain of a protein capable of signaling the recognition of a pathogen-associated molecular domain (PAMP) and activating innate immune gene expression; and
c) a linker positioned between (a) and (b), said linker permitting signal transduction from the EC domain to the IC domain, hereby activating innate immune gene expression, wherein the EC domain and the IC domain are not from the same naturally existing protein.

19. A method for triggering innate immune response, comprising:
a) administering to a subject a composition comprising an expression vector comprising the polynucleotide of claim 1, wherein after administration, the chimeric protein is expressed as a transmembrane protein on cell surface of certain cells of the subject, and
b) allowing the extracellular domain of the chimeric protein to bind to a pathogen to which the subject is exposed, thereby triggering innate immune response.

20. The method of claim 19, wherein the composition is administered intra-nasally or through a part of the respiratory tract in a form of nebulization.

21. The method of claim 19, wherein the composition is administered to the subject in a lipid particle (LNP).

22. The method of claim 19, wherein the polynucleotide has a nucleotide sequence of SEQ ID NO: 2.

23. The method of claim 19, wherein the polynucleotide encodes a chimeric protein having an amino acid sequence of SEQ ID NO: 1.

24. The method of claim 19, wherein the polynucleotide is a DNA or an mRNA and the composition further comprises an ingredient that stabilizes the DNA or mRNA.

25. The method of claim 24, wherein the composition further comprises an ingredient that helps the DNA or mRNA enter into cells of the subject.


SEQUENCE LISTING (4 sequences)

SEQ ID NO: 1 - protein, 981 aa, synthetic construct
GATATCMSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGGGGSGGGGSGGGGSGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVHTCTAGA

SEQ ID NO: 2 - DNA, 2919 bp, synthetic construct
  source 1..2919: mol_type = other DNA
gatatcatgagcagcagcagctggctgctgctgagcctggtggccgtgaccgccgctcagagcaccatcgaggagcaagccaagaccttcctggacaagttcaaccacgaggccgaggacctgttctatcagagcagcctggctagctggaactacaacaccaacatcaccgaggagaacgtgcagaacatgaacaacgccggcgacaagtggagcgccttcctgaaggagcagagcaccctggctcagatgtaccccctgcaagagattcagaacctgaccgtgaagctgcagctgcaagccctgcagcagaacggcagcagcgtgctgagcgaggacaagagcaagagactgaacaccatcctgaacaccatgagcaccatctacagcaccggcaaggtgtgcaaccccgacaacccccaagagtgcctgctgctggagcccggcctgaacgagatcatggccaacagcctggactacaacgagagactgtgggcctgggagagctggagaagcgaggtgggcaagcagctgagacccctctacgaagaatacgtggtgctgaagaacgagatggctagagccaaccactacgaggactacggcgactactggagaggcgactacgaggtgaacggcgtggacggctacgactacagcagagggcagctgatcgaggacgtggagcacaccttcgaggagatcaagccgctctacgagcacctacacgcctacgtgagagccaagctgatgaacgcctaccctagctacatcagccccatcggctgcctgcccgcccacctgctgggcgacatgtggggcagattctggaccaacctgtacagcctgaccgtgcccttcgggcagaagcccaacatcgacgtgaccgacgccatggtggaccaagcctgggacgctcagagaatcttcaaggaggccgagaagttcttcgtgagcgtgggcctgcccaacatgacccaaggcttctgggagaacagcatgctgaccgaccccggcaacgtgcagaaggccgtgtgccaccccaccgcctgggacctgggcaagggcgacttcagaatcctgatgtgcaccaaggtgaccatggacgacttcctgaccgcccaccacgagatgggccacattcagtacgacatggcctacgccgctcagcccttcctgctgagaaacggcgccaacgagggcttccacgaggccgtgggcgagatcatgagcctgagcgccgccacccccaagcacctgaagagcatcggcctgctgagccccgacttccaagaggacaacgagaccgagatcaacttcctgctgaagcaagccctgaccatcgtgggcaccctgcccttcacctacatgctggagaagtggagatggatggtgttcaagggcgagatccccaaggatcagtggatgaagaagtggtgggagatgaagagagagatcgtgggcgtggtggagcccgtgccccacgacgagacctactgcgaccccgctagcctgttccacgtgagcaacgattactccttcataagatactacacaagaaccctgtatcagtttcagttccaagaggccctgtgccaagccgccaagcacgagggccccctgcacaagtgcgacatcagcaactcgaccgaggccgggcagaagctgttcaacatgctgagactgggcaagagcgagccctggaccctggccctggagaacgtggtgggcgccaagaacatgaacgtgagacccctgctgaactacttcgagcccctgttcacctggctgaaggatcagaacaagaacagcttcgtgggctggagcaccgactggagcccctacgccgatcagagcatcaaggtgagaatcagcctgaagagcgccctgggcgacaaggcctacgagtggaacgacaacgagatgtacctgttcagaagcagcgtggcctacgccatgagacagtacttcctgaaggtgaagaatcagatgatcctgttcggcgaggaggacgtgagagtggccaacctgaagccccgtatcagcttcaacttcttcgtgaccgcccccaagaacgtgagcgacatcatccctagaaccgaggtggagaaggccatcagaatgagcagaagcagaatcaacgacgccttcagactgaacgacaatagcctggaatttctgggcattcagcccaccctgggcccccccaatcagccccccgtgagcatctggctgatcgtgttcggcgtggtgatgggcgtgatcgtggtgggcatcgtgatcctgatcttcaccggcatcagagacagaaagaaaaagaacaaggctagatcgggcgggggtggcagtggtgggggtgggagcggcggcggaggatccggctggcgcatcagcttctattggaacgtgagcgtgcacagagtgctgggcttcaaggagatcgacagacagaccgagcagttcgagtacgccgcctacatcatccacgcctacaaggacaaggactgggtgtgggagcacttcagcagcatggagaaggaggatcagagcctgaagttctgcctggaggagagagacttcgaggccggcgtgttcgagctggaggccatcgtgaacagcatcaagagaagcagaaagatcatcttcgtgatcacccaccacctgctgaaggaccccctgtgcaagagattcaaggtgcaccacgccgtgcagcaagccatcgagcagaacctggacagcatcatcctggtgttcctggaggagatccccgactacaagctgaaccacgccctgtgcctgagaagaggcatgttcaagagccactgcatcctgaactggcccgtgcagaaggagagaatcggcgccttcagacacaagctgcaagtggccctgggcagcaagaacagcgtgcactctaga

SEQ ID NO: 3 - protein, 46 aa, synthetic construct
AEAAAKEAAAKEAAAKEAAAKALEAEAAAKEAAAKEAAAKEAAAKA

SEQ ID NO: 4 - protein, 15 aa, synthetic construct
GGGGSGGGGSGGGGS